(19) United States
(12) Patent Application Publication
(10) Pub. No.: US 2005/0121360 A1
(43) Pub. Date: Jun. 9, 2005

(54) Traction fluids by coupling of cyclic hydrocarbon monomers with olefins

(75) Inventors: Richard M. Lange, Euclid, OH (US); Leonard E. Orzech JR., Brunswick, OH (US)

Correspondence Address: THE LUBRIZOL CORPORATION;ATTN: DOCKET CLERK, PATENT DEPT., 29400 LAKELAND BLVD., WICKLIFFE, OH, 44092, US

(73) Assignee: The Lubrizol Corporation, Wickliffe, OH (US)

(21) Appl. No.: 10/730,685

(22) Filed: Dec. 8, 2003

(51) Int. Cl. C10M 105/02; C07C 2/74

(52) U.S. Cl. 208/14; 585/255; 585/1; 585/2

(57) ABSTRACT

A composition suitable for use as a traction fluid comprises a substantially completely hydrogenated addition product of at least one olefin monomer containing a cyclic hydrocarbon structure, with at least one non-cyclic olefin monomer of at least 4 carbon atoms.

### BACKGROUND OF THE INVENTION

The present invention relates to new fluids which can be prepared by coupling cyclic hydrocarbon monomers with non-cyclic olefins. The fluids can be used alone or in combination with other components as traction fluids, that is, as lubricants for mechanical transmissions including continuously variable transmissions.

Continuously-variable transmissions (“CVT”s), both push-belt and toroidal types, are becoming more important as automotive power-transmitting devices, with the ability to replace the standard automatic transmissions commonly used in power trains. The CVTs can eliminate the grabbing problems associated with traditional multigear automatic-shifting transmissions that result in jerky shift feel. CVTs provide for smooth power transition over a theoretically infinite speed range, and can be useful in mechanical devices which involve speed control.

But CVTs, particularly the toroidal types, require a special type of frictional fluid for proper operation without power loss. These are commonly called “traction fluids,” since they exhibit a relatively high angular frictional component during operation, with a high value of sliding-to-rolling friction, which enables angular energy transfer smoothly from the drive side of a CVT transmission, to the driven side.

The literature contains a large number of domestic and foreign patents from a number of companies, dating from the late 1950's and early 1960's, that describe various structurally-different types of fluids. Some of the earliest include hydrocarbon fluids such as the hydrogenated a-methylstyrene dimers and phenylindenes from Monsanto, as well as a variety of other saturated, small-ring-containing (i.e., cyclopentyl, cyclohexyl, cycloheptyl) compositions. Saturated bicyclic hydrocarbons (e.g., alkyl-substituted norbornanes) have also been claimed as useful traction fluids.

Aside from certain ester compositions, the overwhelming majority of patented solely-hydrocarbon traction fluids appear to be derived by hydrogenation of various types of coupled aromatic intermediates, the most-used of which have been α-methylstyrene dimers. The aromatic rings of linear dimers of α-methyl-styrene have been hydrogenated to provide efficient traction fluids at high temperatures, but the resulting saturated alicyclic products tend to be extremely viscous liquids, or even solids, at temperatures below about −10° C. A commercial product comprising this type of hydrogenated α-methyl-styrene dimer, sold commercially as Santotrac 20™, has a −30° C. Brookfield viscosity of about 70 Pa·s (70,000 cP) or greater, and a −40° C. viscosity of 300 Pa·s (300,000 cP) or greater.

The common feature of patented hydrocarbon traction fluids is that they contain alicyclic (ring) structures in their molecular compositions. Measured Traction Coefficients (TCs) are high, in the range of 0.085 to 0.10 (measured at about 100-125° C.) for many of these fluids. In comparison, refined mineral oils generally range from about 0.015 to 0.025 for paraffinic oils, and from about 0.04 to 0.06 for certain hydrotreated naphthenic oils. The alicyclic moieties in the traction fluid molecules are conducive to “packing,” or association, particularly at lower temperatures, which makes them good traction fluids, but also results in the problem that they have exceedingly high viscosities at low temperatures, particularly below about 0 to 10° C.

In the case of styrenic derived traction fluids, α-methylstyrene (α-MS) dimers provided the earliest traction fluids, supplied by Monsanto. Both linear and cyclic dimers can be formed on dimerization, and early literature stressed the importance of obtaining the linear dimer for the final product. Reduction of the aromatic rings in the linear dimers is believed to provide the traction performance, while the aliphatic portions of the reduced molecules are believed to provide the fluidity characteristics. Traction coefficients (TC) for this type of product are high, on the order of about 0.10. However, one problem with making α-MS derived fluids is that its linear dimers have a tendency to cyclize and form phenylindane; when the indane is reduced, the saturated alicyclic product is a cyclohexyl-substituted perhydroindane, which has a freezing point near 15° C. Although the TC is also high for this product (on the order of about 0.10), any low temperature fluidity derived from the usual aliphatic “backbone” in the dimer structure is lost because of the rigidity of the fused ring system in the molecule.

Thus, for example, U.S. Pat. No. 3,975,278, Wygant, Aug. 17, 1976, discloses tractants comprising hydrogenated linear dimers of α-alkyl styrene.

U.S. Pat. No. 3,440,894, Hammann et al., discloses a traction fluid of, e.g., cyclodecane, bicyclohexyl, 1,2-tercyclohexyl, dicyclohexylmethane, and others, including alkyl-substituted cycloalkanes in which the alkyl group can be a normal or branched alkyl radical.

U.S. Pat. No. 3,925,217, Green et al., Dec. 9, 1975, discloses lubricants for rolling contact bearing, comprising cyclohexyl compounds having two or more cyclohexyl rings, being fused, concatenated or linked by, e.g., one or more C₁ to C₁₆ alkylene linkages. Preferred materials are hydrogenated linear or cyclic dimers or trimers of alpha-methylstyrene and alpha-ethylstyrene. Listed examples of other linked cyclohexyl compounds include (among many others) x-isohexyl-4′-isopropyldicyclohexyl and 2,3-dicyclohexyl-2,3-dimethylbutane.

U.S. Pat. No. 4,704,490, Tsubouchi et al, Nov. 3, 1987, discloses a fluid for traction drive containing (A) an alkene derivative having at least three cyclohexane rings in a molecule, and (B) an alkane derivative having a main chain of two or three carbon atoms, to which at least two methyl groups are bonded, each having two cyclohexane rings in a molecule each bonded to one of the terminal carbon atoms of the alkane.

U.S. Pat. No. 4,922,047, Chen et al., May 1, 1990, discloses traction fluids from bicyclic and monocyclic terpenes with zeolite catalyst. The reaction feed can also be mixed with a light olefin, e.g., propylene and/or butylenes.

U.S. Pat. No. 4,975,215, Abe et al., Dec. 4, 1990, discloses a traction fluid from dimer, trimer, or polymer of cyclic monoterpenoid monomers. For example, limonene is reacted in the presence of active clay, then hydrogenated.

The present invention permits the use of readily available materials, such as vinyl aromatic compounds and natural terpenes, in combination with non-cyclic olefins, to produce useful hydrocarbon traction fluids having very high traction coefficients, with significantly improved (i.e., lower) viscosities at −30° to −40° C.

A further advantage is obtained when terpenes are used as the starting materials, in that the resulting products are significantly easier to hydrogenate than are aromatic rings, permitting use of more moderate temperatures and hydrogen pressures and avoiding the necessity for expensive catalysts in the synthesis.

### SUMMARY OF THE INVENTION

The present invention provides a composition suitable for use as a traction fluid, comprising a substantially completely hydrogenated addition product of (a) at least one olefin monomer containing a cyclic hydrocarbon structure, with (b) at least one non-cyclic olefin monomer of at least 4 carbon atoms, provided that if the olefin monomer is monounsaturated, then it contains at least 5 carbon atoms.

The invention also provides a method for lubricating a power transmission apparatus, comprising employing therein the above-mentioned composition.

The invention also provides a method for preparing a composition suitable for use as a traction fluid, comprising:
(a) combining
   (i) at least one olefin monomer containing a cyclic hydrocarbon structure, with
   (ii) at least one non-cyclic olefin monomer of at least 4 carbon atoms, provided that if the olefin monomer is mono-unsaturated, then it contains at least 5 carbon atoms; and
   (iii) an acid catalyst;
(b) maintaining the resulting mixture at about 25° C. to about 150° C. for a time sufficient to permit reaction of components (a)(i) and (a)(ii);
(c) optionally removing the volatile components from the product of (b); and
(d) substantially completely hydrogenating the resulting reaction product.

### DETAILED DESCRIPTION OF THE INVENTION

Various preferred features and embodiments will be described below by way of non-limiting illustration.

The traction fluid of the present invention comprises an addition product of unsaturated monomers (a) and (b). Such products are normally prepared by the addition reaction of the double bonds of the monomers. Occasionally the product may be referred to as a condensation product, although more usually that term is reserved for products formed with the concurrent elimination of a small molecule such as water.

The first monomer, (a), is an olefin monomer containing a cyclic hydrocarbon structure. The cyclic moiety can be either aromatic or alicyclic. For the purposes of the present invention, both types may be considered substantially equivalent, since after the resulting addition product is hydrogenated, very similar structures will normally be obtained. The cyclic olefin monomer, in either case, will typically contain a vinyl group or a substituted vinyl group, for example, bearing a substituent at the α position.

Aromatic olefins can be monocyclic or polycyclic monomers, that is, containing one or more benzene rings, as well as naphthalene nuclei and higher condensed ring structures. Among these, the substituted benzenes are preferred. The aromatic monomer is preferably a vinyl arene, that is, a monomer containing a vinyl group or a substituted vinyl group which is attached to the aromatic ring. In particular, vinyl arenes such as styrene and various substituted styrenes, are desirable because of their ready availability. Such materials include α-methylstyrene and ring-alkylated styrenes such as 2- and 4-alkylstyrenes, e.g., 4-t-butylstyrene or 2-methyl-styrene.

Substituted styrenes can be generally depicted by the following structure, 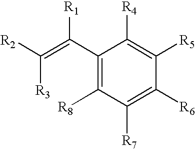
wherein each of the R groups is hydrogen or a hydrocarbyl group. In one embodiment R₂ and R₃ are hydrogen, in order that the double bond is not excessively sterically hindered and thus is capable of readily undergoing the addition reaction with monomer (b). R₁ is commonly hydrogen or methyl. R₄ and R₈ are likewise preferably hydrogen, and R₅, R₆, and R₇ are hydrogen or alkyl groups. The hydrocarbyl groups substituted on the benzene ring typically contain 1 to 12 carbon atoms each, e.g., 2 to 8 carbon atoms. The total number of carbon atoms in the aromatic monomer is typically 8 to 30, preferably 9 to 20 or 9 to 18 or 10 to 12 (or combinations of these limits such as 8-20, 8-18, 8-12).

The vinyl aromatic compound can also contain more than one vinyl group attached to the aromatic structure, such as divinylbenzene or divinylnaphthalene. A divinylbenzene would be represented in the above structure in which one of R₄ to R₈ is —C(R₁)═CR₂R₃. Thus, the aromatic monomer may contain 1, 2, or 3 olefinic double bonds, typically 1 or 2 olefinic double bonds, and more commonly one double bond.

Another type of olefin monomer containing a cyclic hydrocarbon structure is a cyclic terpene. Examples of cyclic terpenes include α-pinene, β-pinene, limonene (a preferred material), α-terpinene, β-terpinene, and β-phellandrene. Limonene, in particular, has a carbon skeleton structure which is the same as that of a styrene and, upon hydrogenation, its addition product will be very similar to those obtained from styrenes.

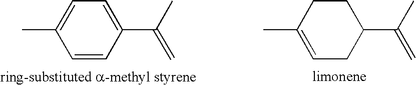

Limonene, to be sure, also contains a non-aromatic double bond within the ring which is capable of undergoing addition with other monomers in a way that the aromatic ring of styrene cannot. However, it is believed that the majority of the addition reaction of limonene will occur at the exterior, and less sterically hindered, double bond. In the case of limonene, the presence of the substituted vinyl group is especially desirable. The presence of an α-substituent on the vinyl group is desirable in this and other cyclic monomers, providing for generally good reactivity.

Cyclic terpenes typically contain 1 or 2 olefinic double bonds, although structures with 3 or more double bonds are envisioned. The monocyclic terpenes typically contain 2 double bonds, and the bicyclic terpenes (the pinenes) typically contain 1 double bond. The cyclic terpenes can contain, variously, double bonds within the ring structure (e.g., α-pinene, α-terpinene, β-terpinene, and β-phellandrene); and some contain double bonds attached to, but external to the ring, such as vinylidene groups, other than vinyl groups (e.g., β-pinene, β-terpinene, β-phellandrene). The variations in the location of the olefinic double bonds will, in each case, lead to some variation in the structure of the resulting addition product, but in all cases the cyclic nature of the initial monomer will normally be retained. The cyclic terpenes generally contain 10 carbon atoms, particularly non-substituted terpenes, although hydrocarbyl-substituted terpenes would also be acceptable and are included within the general scope of the invention. These contain a larger number of carbon atoms, for instance, up to 24, or to 20, or to 18, or to 12 carbon atoms.

Other suitable olefin monomers containing a cyclic hydrocarbon structure, although not cyclic terpenes, include such monomers as 4-vinylcyclohexene and trivinylcyclohexene.

The second monomer (b) used in preparing the present compositions is at least one non-cyclic olefin monomer of at least 4 carbon atoms, provided that if the olefin monomer is monounsaturated, then it contains at least 5 carbon atoms. Preferably the monomer (b), in either case, contains at least 5 carbon atoms, and more preferably 6 to 24 or 6 to 18, or 6 to 12, or 8 to 10 carbon atoms.

The olefin monomer of (b) is a non-cyclic olefin monomer, in contrast with the cyclic monomers of (a). That is, it does not contain an aromatic or alicyclic ring structure, but has an “open” structure. This olefin can be linear or branched. It can contain 3 or more double bonds, but typically contains 1 to 3, or 1 to 2, or a single ethylenic double bond.

Examples of suitable non-cyclic olefin monomers include monoolefins such as pentenes, including 2-methyl-butene, linear and branched hexenes including “neohexene,” linear and branched octenes, linear and branched decenes, propylene trimers, propylene tetramers, isobutylene dimers, trimers, and tetramers, such as diisobutylene (CH₂═C(CH₃)—CH₂—C(CH₃)₂—CH₃ and isomers thereof). Examples of suitable non-cyclic dienes include butadiene, isoprene, 1,3-pentadiene, and 1,3 hexadiene. Other suitable non-cyclic olefins include non-cyclic terpenes such as myrcene (a branched triene containing 11 carbon atoms). Also suitable are various commercial mixtures of linear and branched olefins comprising odd and even carbon numbers, made by, e.g., the “SHOP” process, that is “Shell Heavy Olefins Process,” based on olefin metathesis, using ethylene to produce terminal olefins, available from Shell.

Monomers (a) and (b) are typically reacted in an addition reaction to form a dimer or oligomer. The oligomer will typically contain up to 10 total units of the (a) and (b) monomers, preferably up to 6 or up to 4 units, and in one case will be a dimer, containing one unit of (a) and one unit of (b). The reaction product will frequently be a mixture of individual dimers and oligomers of varying lengths and varying compositions. Components (a) and (b) will each typically comprise 10 to 90 weight percent of the product, preferably 15 to 85, or 20 to 80, or 30 to 70, or 40 to 60 percent, expressed as percent by weight of the total of all monomers present in the addition product. In one embodiment, component (a) is a vinyl aromatic monomer, and the amount of (a) is 40 to 80 (e.g., 50 to 70) percent by weight of the all the monomers in the addition product, and the amount of monomer (b) is 60 to 20 (e.g., 50 to 30) percent by weight of all the monomers in the addition products. When the product is substantially the dimeric product, it will typically be the product of one mole of (a) with one mole of (b), the resulting dimer being the 1:1 mole adduct. Other materials, such as the 1:1 addition product of two moles of (a) and the 1:1 addition product of two moles of (b) may also be present in varying amounts.

The addition product can be prepared by the acid-catalyzed addition reaction of the monomers of (a) and (b). Suitable acid catalysts include Brönsted and Lewis acid catalysts, including sulfur acids, phosphorus acids, and halogen acids. Examples include sulfonated crosslinked polystyrene resins of the types known as Amberlite™ and Amberlyst™ (from Rohm & Haas), sulfuric, sulfonic, phosphoric, polyphosphoric, phosphorous, phosphonic, and phosphinic acid and other strong mineral acids and derivatves thereof such as hydrochloric acid; phosphorus halides, heteropolyacid catalysts, aluminum halides, titanium halides, boron trifluoride, hydrogen fluoride, acidic zeolites, and strongly acidic clays such as montmorillonite K-10, and various mixtures of these. The amount of the catalyst used for the addition reaction is typically 0.5 to 4.0 percent by weight of the reaction mixture, more commonly 1 to 2 percent. The catalysts, particular those in solid form, can typically be reused or recycled.

Heteropolyacid catalysts are known from, e.g., U.S. Pat. No. 6,346,585, and include a variety of complex oxoanions and their corresponding acids and salts, of which H₃PW₁₂O₄₀ is typical. Heteropolyacid catalysts are active both as their acid form, in their fully salted form, in which the hydrogen ions are replaced by other cations such as metal or ammonium ions, and in their partially salted forms, in which a portion of the hydrogen ions have been thus replaced. Particularly useful heteropolyacid catalysts include those of the structure (NH₄)_{2.5}H_{0.5}PW₁₂O₄₀. For more information, attention is directed to Misono, “Heterogeneous Catalysis by Heteropoly Compounds of Molybdenum and Tungsten,” Catal. Rev.-Sci. Eng., 29 (2&3), 269-321 (1987), in particular, pages 270-280. The heteropolyacids may be used as 100% solids, or they may be supported on suitable inorganic substrates.

The addition reaction can be conducted in a relatively inert solvent, typically a hydrocarbon solvent. Examples of suitable solvents include linear, branched, or cyclic saturated paraffins, benzene and certain C1 to C6 alkyl-substituted aromatic hydrocarbons such as toluene, cumene, and ethylbenzene, cyclohexane, alkylcyclopentanes, alkylcyclohexanes, diphenyl oxides, alkylated diphenyl oxides, diphenylalkkanes, and halogenated solvents such as methylene chloride (although halogen contamination of the resulting products may desirably be avoided). A preferred class of solvents are those which can act as chain-transfer agents by hydride abstraction in the cationic condensation process of the present invention, thus helping to minimize the formation of higher molecular weight oligomers or polymers that might detract from improved low temperature viscometrics. The amount of the solvent can typically be 10 to 70 percent by weight of the reaction mixture, preferably 20 to 60 percent or 30 to 50 percent.

The addition reaction can occur at any of a variety of temperatures including room temperature or elevated temperatures, typically 20° C. to 180° C., or 25 to 250° C., or 40 to 120° C. or 50 to 110° C. The temperature will depend to some extent on the particular monomers employed: for limonene and diisobutylene, temperatures of 110 to 135° C. can be desirable; for reactions with vinyl arenes, somewhat lower temperatures can be useful. Lower temperatures, however, can favor homopolymerization of vinyl arenes, which is not normally desired, but may be useful in producing products of higher viscosity. Relatively higher temperatures favor termination and chain transfer reactions, and form ation of the desirable (a)(b) dimer and mixed trimers. Chain-transfer solvents can be particularly useful in limiting molecular weight of the coupling products. The components are reacted, typically with stirring for a time sufficient to permit complete or substantially complete reaction of the monomers. Suitable times will, of course, depend to some extend on the temperature selected, but may be in the range of ½ to 8 hours, or 1 to 6 hours, 1 to 3 hours, or 2 to 4 hours.

The solvent can be removed, if desired, after the addition reaction, or it can be retained during the subsequent hydrogenation step. Indeed, a portion of the solvent may become a part of the final traction fluid product, particularly if it substantively enters into the addition reaction by virtue of its activity as a chain transfer agent. Removal of the solvent, along with other volatile components such as unreacted starting material, can be effected by stripping or purging with inert gas, optionally under vacuum.

The resulting reaction product, which is typically a mixture of various materials, generally containing unsaturation, is subjected to hydrogenation. The hydrogenation should be sufficiently vigorous to substantially completely remove unsaturation from the product. The unsaturation to be removed includes both residual olefinic unsaturation as well as the aromatic unsaturation present if an aromatic material is used as component (a). The resulting product will be “substantially completely” hydrogenated, by which it is meant that at least 90 percent of the residual unsaturation initially present in the addition product is removed, for instance, 92 to 94%, or even a larger amount such as 96% or 98% or 99%.

Hydrogenation can be effected using hydrogen or a hydrogen-providing source and a suitable catalyst. Typical catalysts include free or supported metal catalysts, where the metal can be, among others, platinum, palladium, rhodium, or nickel. Rhodium is particularly suitable for hydrogenation of aromatic materials; nickel is satisfactory for reduction of non-aromatic materials derived from terpenes. The metal is typically provided on a support such as alumina, silica, carbon, or kieselguhr. A catalyst of 5% rhodium on carbon is useful for hydrogenation of the aromatic products. The hydrogenation typically is conducted under pressure in an autoclave (e.g., stainless steel) at up to 6.9 MPa (gauge) (1000 psig), typically 2.1 to 3.4 MPa (300-500 psig) and at a temperature of 100-160° C., e.g., 120-130° C. The amount of time will depend on the nature and chemical structure of the materials being hydrogenated and on the type and amount of the catalyst. At a typical level of about 2 g catalyst (including support) per 100 g of material to be reduced, the products from terpenes may be hydrogenated for 4 to 10 hours or 6 to 8 hours using a nickel catalyst. Under similar conditions, the products from aromatic materials may be hydrogenated for 8 to 20 hours, or 10 to 16 hours, or 12 to 14 hours using a supported rhodium catalyst. Effective completion of the reaction can be monitored by IR or NMR spectroscopy, and is typically achieved when 95-97% of the unsaturation is removed. Determination of specific conditions can be readily determined by the person skilled in the art. After the hydrogenation reaction, the catalyst may be recovered and reused.

Depending on the monomers used and the processing parameters employed, the resulting hydrogenated condensation product will typically have a kinematic viscosity of 2.5 to 8 mm²/s (cSt) at 100° C. and a Brookfield viscosity (ASTM D2983) of less than 70 Ps-s (70,000 cP), preferably less than 50, or 35, or even 10 Pa-s (50,000, 35,000, or 10,000 cP) at −30° C. The viscosity can be controlled to a large extent by selection of the monomers and reaction conditions (to minimize higher oligomer formation, for instance). It is believed that the presence of the non-cyclic olefin monomer (b) in the coupled products is in large measure responsible for the comparative low viscosity at low temperatures. The fluids of this invention can have traction coefficients of 0.092 to 0.10, more commonly 0.094 to about 0.098.

Traction fluids prepared from the products of the present invention may include blends of a plurality of such hydrogenated addition products having differing viscosities, selected so as to provide a material having the desired low temperature viscosity performance, and desirably also suitable high temperature properties. Traction fluids of the present invention may also include an oil of lubricating viscosity of any of a variety of types, including other types of traction fluids, in order to obtain the desired properties. However, care should be employed in order that the amount of additional oils (which may not exhibit such excellent traction performance) should normally be limited so that the traction coefficient of the resulting mixture does not drop below 0.085, preferably 0.090 or 0.095. The products of the present invention can also be mixed with one or more other traction fluids, several of which are available commercially.

The traction fluids of the present invention can be formulated into complete lubricant formulations by including other additives suitable for providing the desired functionality to the fluid. Thus, a complete fluid can contain at least one additive selected from the group consisting of dispersants, detergents, friction modifiers, antioxidants, metal passivators, viscosity modifiers, and antiwear agents, as well as other materials such as seal swell agents, corrosion inhibitors, dyes, and foam inhibitors, in an amount sufficient to improve the performance of said composition in a power transmission device. These materials and their preparation are described in greater detail in PCT Patent Application WO 01/34738 and in the references cited therein.

The dispersants useful as a component in the present fluids include acylated amines, carboxylic esters, Mannich reaction products, hydrocarbyl substituted amines, and mixtures thereof. Acylated amine dispersants include reaction products of one or more carboxylic acylating agent and one or more amine. Hydrocarbyl-substituted maleic acylating agents are the preferred unsaturated acylating agent. The procedures for preparing the acylating agents are well known to those skilled in the art and have been described for example in U.S. Pat. No. 3,412,111. The amines which react with the acylating agents may be known amines, preferably a polyamine, such as an alkylenepolyamine or a condensed polyamine. Polyamines can be aliphatic, cycloaliphatic, heterocyclic or aromatic. Examples of the polyamines include alkylene polyamines, hydroxy containing polyamines, arylpolyamines, and heterocyclic polyamines.

Carboxylic ester dispersants can be prepared by reacting at least one or more carboxylic acylating agents, preferably a hydrocarbyl substituted carboxylic acylating agent, with at least one organic hydroxy compound and optionally an amine. The hydroxy compound may be an alcohol or a hydroxy containing amine.

In another embodiment, the dispersant can be a hydrocarbyl-substituted amine. These hydrocarbyl-substituted amines are well known to those skilled in the art. Typically, hydrocarbyl substituted amines are prepared by reacting olefins and olefin polymers, including the above polyalkenes and halogenated derivatives thereof, with amines (mono- or polyamines).

In another embodiment, the dispersant can be a Mannich dispersant. Mannich dispersants are generally formed by the reaction of at least one aldehyde, such as formaldehyde and paraformaldehyde, at least one amine, preferably a polyamine, such as a polyalkylenepolyamine, and at least one alkyl substituted hydroxyaromatic compound. Other suitable dispersants include dispersants made by acid-catalyzed glyoxylation of olefins and polyolefins using glyoxylic acid, or a derivative thereof, subsequently condensed with polyamines.

The dispersant can also be a dispersant which has been treated or reacted with any of a variety of common agents. That is, they can be borated dispersants or sulfurized dispersants, or metal-containing dispersants.

The amount of the dispersant in the traction fluid composition, if present, is preferably 1 to 10 weight percent, preferably 1.5 to 7 weight percent, and more preferably 2 to 3 weight percent.

The additive component for the traction fluid can also contain one or more detergents, which are normally salts, and specifically overbased salts. Overbased salts, or overbased materials, are single phase, homogeneous Newtonian systems characterized by a metal content in excess of that which would be present according to the stoichiometry of the metal and the particular acidic organic compound reacted with the metal. The overbased materials are prepared by reacting an acidic material (typically an inorganic acid or lower carboxylic acid, preferably carbon dioxide) with a mixture comprising an acidic organic compound, a reaction medium comprising at least one inert, organic solvent (such as mineral oil, naphtha, toluene, xylene) for said acidic organic material, a stoichiometric excess of a metal base, and a promoter.

The acidic organic compounds useful in making the overbased compositions of the present invention include carboxylic acids, sulfonic acids, phosphorus-containing acids, phenyls or mixtures thereof. Preferably, the acidic organic compounds are carboxylic acids or sulfonic acids with sulfonic or thiosulfonic groups (such as hydrocarbyl-substituted benzenesulfonic acids), and hydrocarbyl-substituted salicylic acids.

The metal compounds useful in making the overbased salts are generally any Group 1 or Group 2 metal compounds (CAS version of the Periodic Table of the Elements). The Group 1 metals of the metal compound include Group 1a alkali metals (e.g., sodium, potassium, lithium) as well as Group 1b metals such as copper. The Group 1 metals are preferably sodium, potassium, lithium and copper, preferably sodium or potassium, and more preferably sodium. The Group 2 metals of the metal base include the Group 2a alkaline earth metals (e.g., magnesium, calcium, strontium, barium) as well as the Group 2b metals such as zinc or cadmium. Preferably the Group 2 metals are magnesium, calcium, barium, or zinc, preferably magnesium or calcium, more preferably calcium.

The amount of the overbased material, that is, the detergent, if present, is preferably 0.05 to 5 percent by weight of the composition, more preferably 0.05 to 3 percent, 0.1 to 1.5 percent, or most preferably 0.2 to 1 percent by weight.

Both a dispersant and a detergent can be included in the composition. For example, a succinimide dispersant and a calcium overbased sulfonate detergent can be used.

The compositions of the present invention can also contain a viscosity index modifier, also known as a viscosity modifier, typically a polymeric viscosity index modifier, preferably in limited amounts, that is, up to 10 percent by weight of the composition. In one embodiment, the amount of this component is 0 to 3 percent by weight, and in one embodiment the traction fluids are substantially free from polymeric viscosity index modifiers. Polymeric viscosity index modifiers (VMs) are extremely well known in the art and most are commercially available. Hydrocarbon VMs include polybutenes, poly(ethylene/propylene) copolymers, isobutylene/isoprene copolymers, optionally hydrogenated, and hydrogenated polymers of styrene with butadiene or isoprene. Ester VMs include esters of styrene/maleic anhydride polymers, esters of styrene/maleic anhydride/acrylate or methacrylate terpolymers, polyacrylates, polymethacrylates, and vinyl acetate-fumarate ester copolymers. Dispersant viscosity modifiers based on any of the foregoing polymers, modified to impart dispersant functionality, are also useful. The polymethacrylates are available from RohMax and from The Lubrizol Corporation; polybutenes from Ethyl Corporation, BASF, and Lubrizol; ethylene/propylene copolymers from ExxonMobil and ChevronTexaco; hydrogenated polystyrene/isoprene polymers from Shell; styrene/maleic esters and vinyl acetate/fumarate esters from Lubrizol, and hydrogenated styrene/butadiene polymers from BASF.

Antiwear agents, another optional component, include metal salts of a phosphorus acid. Metal salts of the formula [(R⁸O)(R⁹O)P(═S)S]ₙM, are readily obtainable by the reaction of phosphorus pentasulfide (P₂S₅) and an alcohol or phenyl to form an O,O-dihydrocarbyl phosphorodithioic acid corresponding to the formula (R⁸O)(R⁹O)P(═S)SH. The reaction involves mixing at a temperature of 20° C. to 200° C., four moles of an alcohol or a phenyl with one mole of phosphorus pentasulfide. Hydrogen sulfide is liberated in this reaction. The acid is then reacted with a basic metal compound to form the salt. The metal M, having a valence n, generally is aluminum, lead, tin, manganese, cobalt, nickel, zinc, or copper, and most preferably zinc. The basic metal compound is thus preferably zinc oxide, and the resulting metal compound is represented by the formula [(R⁸O)(R⁹O)P(═S)S]₂Zn. The R⁸ and R⁹ groups are independently hydrocarbyl groups that are preferably free from acetylenic and usually also from ethylenic unsaturation. They are typically alkyl, cycloalkyl, aralkyl or alkaryl group and have 3 to 20 carbon atoms, preferably 3 to 16 carbon atoms and most preferably up to 13 carbon atoms, e.g., 3 to 12 carbon atoms. The alcohol which reacts to provide the R⁸ and R⁹ groups can be a mixture of a secondary alcohol and a primary alcohol, for instance, preferably a mixture of isopropanol and 4-methyl-2-pentanol. Such materials are often referred to as zinc dialkyldithiophosphates or simply zinc dithiophosphates. They are well known and readily available to those skilled in the art of lubricant formulation. The amount of the metal salt of a phosphorus acid in a completely formulated lubricant, if present, will typically be 0.1 to 4 percent by weight, preferably 0.5 to 2 percent by weight, and more preferably 0.75 to 1.25 percent by weight. Its concentration in a concentrate will be correspondingly increased, to, e.g., 5 to 20 weight percent.

Other phosphorus compounds can also be present, such as a phosphorus acid, a phosphorus acid salt, a phosphorus ester, or mixtures thereof. The phosphorus acid or ester can be of the formula (R¹X)(R²X)P(X)ₙXₘR³ or a salt thereof, where each X is independently an oxygen atom or a sulfur atom, n is 0 or 1, m is 0 or 1, m+n is 1 or 2, and R¹, R², and R³ are hydrogen or hydrocarbyl groups, and preferably at least one of R¹, R², or R³ is hydrogen. These R groups can be, specifically, alkyl, phenyl, or alkylphenyl groups. This component thus includes phosphorous and phosphoric acids, thiophosphorous and thiophos-phoric acids, as well as phosphite esters, phosphate esters, thiophosphite esters, and thiophosphate esters. Phosphoric acid and phosphorous acid are well-known items of commerce. Thiophosphoric acids and thiophosphorous acids are likewise well known and are prepared by reaction of phosphorus compounds with elemental sulfur or other sulfur sources. The amount of the above phosphorus acid, salt, or ester in the traction fluid of the present invention, if present, is preferably an amount sufficient to provide at least 0.01 percent by weight of phosphorus to the fluids (calculated as P), preferably 0.01 to 0.1 percent, and more preferably 0.03 to 0.06 or 0.05 percent by weight.

Another optional species in the traction fluids of the present invention is a friction modifier. Friction modifiers include alkoxylated fatty amines, borated fatty epoxides, fatty phosphites, fatty epoxides, fatty amines, borated alkoxylated fatty amines, metal salts of fatty acids, fatty acid amides, glycerol esters, borated glycerol esters, molybdenum compounds such as molybdenum dithiocarbamates, and condensation products of fatty acids and polyamines, including fatty imidazolines. One such material is the condensation product of isostearic acid and diethylene triamine. One preferred example of a friction modifier, zinc salts of fatty acids are well known materials. A preferred acid is oleic acid, and the correspondingly preferred salt is zinc oleate, a commercially available material, the preparation of which is well known and is within the abilities of the person skilled in the art. Slightly basic forms of zinc oleate, represented for example by Zn₄Oleate₆O₁, are also useful. Condensation products of a carboxylic acid with a 1,2 diaminoethane compound are also useful friction modifiers, as are borated epoxides (actually, borate esters), diethoxylated long chain amines, and certain phosphorus-containing materials. The amount of friction modifier, if present, is preferably 0.01 to 2 percent by weight of the traction fluid composition. More preferably it is 0.05 to 1.2 percent, and most preferably 0.1 to 1 percent by weight.

Antioxidants (that is, oxidation inhibitors), including hindered phenolic antioxidants such as 2,6,-di-t-butylphenyl, secondary aromatic amine antioxidants such as dialkyl (e.g., dinonyl) diphenylamine, sulfurized phenylic antioxidants, oil-soluble copper compounds, phosphorus-containing antioxidants, molybdenum compounds such as the Mo dithiocarbamates, organic sulfides, disulfides, and polysulfides. An extensive list of antioxidants is found in U.S. Pat. No. 6,251,840.

The optional low-temperature viscosity control agent (which is to be distinguished from a viscosity index modifier, another optional component described above), which is desirable in certain prior formulations, can often be eliminated entirely from the traction fluids of the present invention, since the present cyclic oligomer inherently has excellent low temperature viscosity properties. However, if an additional low-temperature viscosity control agent is desired, it can be selected from among a variety of materials which are known to be useful for this purpose, including (a) oligomers or polymers of linear alpha olefins of at least 8 carbon atoms, (b) naphthenic oils, (c) synthetic ester oils, (d) polyether oils, (e) alkyl naphthalenes, and mixtures thereof. These materials are distinguishable from the base fluids, described above, in that they are generally lower viscosity materials than the base fluids, typically exhibiting a viscosity of up to or less than 2.5 mm²/s (2.5 cSt), preferably 1.5 to 2.5, or 1.8 to 2.3 mm²/s (1.5 to 2.5 or 1.8 to 2.3 cSt) at 100° C. These are also materials which typically retain a measure of mobility at low temperatures (e.g., −40° C.) and can serve to reduce the low temperature viscosity of fluids to which they are added. Materials which are of unduly high viscosity or which do not retain mobility at low temperatures do not effectively serve as low-temperature viscosity control agents. Determination of viscosity and low temperature mobility is well within the abilities of those skilled in the art. These materials are described in greater detail in PCT Patent Publication WO 01/34738. The amount of the low temperature viscosity control agent in the traction fluid, if present, can be 1 to 20 percent by weight of the traction fluid, or 3 to 15, or 5 to 10 percent by weight.

Metal passivators, which can be used in the traction fluids, include copper passivators, including dimercaptothiadiazoles such as 2,5-bis-alkylthio-1,3,4-thiadia-zoles, e.g., 2,5-bis-nonylthio-1,3,4-thiadiazole and the mono-nonyl analogue. Other metal passivators include triazoles such as benzotriazole, alkyl-substituted benzotriazole, aryl-substituted benzotriazole, and alkylaryl- or arylalkyl-substituted benzotriazole and other substituted benzotriazoles. In one embodiment, the triazole is a benzotriazole or an alkylbenzotriazole in which the alkyl group contains 1 to about 20, or from 1 to about 12, or from 1 to about 8 carbon atoms. 2-Mercaptobenzothiazoles and their derivatives may also be useful.

The compounds and compositions of the present invention can be used in traction power transmission devices, as described above. They can also be used in other applications as gear oils, automatic transmission fluid, including continuously variable transmission fluid, manual transmission fluids (particularly for lubricating a synchronizer in a manual transmission), dual clutch transmission fluid, hydraulic fluids, and other fluids for use in applications for which an increase in coefficient of friction under pressure is desired. The devices in question can be lubricated by supplying the fluids of the present invention thereto, for instance, through a sump or other means.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include:
hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring);
substituted hydrocarbon substituents, that is, substituents containing non-hydrocarbon groups which, in the context of this invention, do not alter the predominantly hydrocarbon nature of the substituent (e.g., halo (especially chloro and fluoro), hydroxy, alkoxy, mercapto, alkylmercapto, nitro, nitroso, and sulfoxy);
hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES 1-17

### Condensations of Vinyl Aromatics with Olefins

### Example 1

A 1-liter four-neck flask is equipped with a mechanical stirrer, thermowell/thermocouple, pressure-equalizing addition funnel with nitrogen inlet, and cold-water condenser. The flask is charged with 300 mL of cumene and 6 grams of dry Amberlyst™ 35 catalyst (Rohm and Haas), and the mixture stirred and heated to 55° C. while purging the system with a slow stream of nitrogen. The addition funnel is charged with a mixture of 150 grams of α-methylstyrene (Lancaster Chemical) and 150 grams of diisobutylene. (Texas Petrochemicals), and the mixture added rapidly drop-wise, at a constant rate, to the warm cumene/catalyst slurry over 45 minutes. The mixture is stirred for 2 hours at 55° C., then filtered to remove catalyst, and stripped at 100° C. at 1.6 kPa (12 mm Hg) pressure to remove volatiles, to yield 282 grams of straw-colored fluid residue. GC/MS analysis of the residue shows a composition comprising:
5.4 wt % unstripped solvent/monomer residues.
9.0 wt % diisobutylene aliphatic dimers
61.2 wt % diisobutylene/α-methylstyrene cross dimers
13.6 wt % phenylindanes (from α-methylstyrene dimerization)
6.8 wt % linear α-methylstyrene dimers
4.0 wt % mixed alkyl-arene trimers

### Example 2

The procedure of Example 1 is followed, but carrying out the reaction at 65° C., with a hold time, after monomer addition, of 3.3 hours. Vacuum-stripping yields a straw-colored residue which, by GC/MS analysis, shows a composition comprising:
5.0 wt % unstripped solvent/monomer residues.
15.6 wt % diisobutylene aliphatic dimers
49.9 wt % diisobutylene/α-methylstyrene cross dimers
26.7 wt % phenylindanes
2.8 wt % mixed alkyl-arene trimers
with no linear α-methylstyrene dimers present.

### Example 3

The procedure of Example 1 is followed, but the reaction was carried out at 95° C., with a hold time of 3.3 hours after monomer addition is complete. Vacuum-stripping yields a straw-colored residue with an analysis (apart from residual monomers) by CG/MS of:
15% diisobutylene aliphatic dimers
40% diisobutylene/α-methylstyrene cross dimers
5% α-methylstyrene dimers
35% α-methylstyrene cyclic dimers, and
5% mixed trimers.

### Example 4

The procedure of Example 1 is followed, using 100 grams of diisobutylene and 200 grams of α-methyl-styrene, with a hold time of 2 hours at 55° C. after monomer addition is complete. Vacuum-stripping yields 279 grams of straw-colored residue which, by GC/MS analysis, shows a composition comprising:
2.5 wt % unstripped solvent/monomer residues.
3.4 wt % diisobutylene aliphatic dimers
49.3 wt % diisobutylene/α-methylstyrene cross dimers
25.9 wt % phenylindanes
9.4 wt % linear α-methylstyrene dimers
9.5 wt % mixed alkyl-arene trimers

### Example 5

The procedure of Example 3 is followed, using 100 grams of diisobutylene and 200 grams of α-methyl-styrene, with a hold time of 3.2 hours at 65° C. after monomer addition is complete. Vacuum-stripping yields 285 grams of straw-colored residue which, by GC/MS analysis, shows a composition comprising:
2.0 wt % unstripped solvent/monomer residues.
4.5 wt % diisobutylene aliphatic dimers
50.1 wt % diisobutylene/α-methylstyrene cross dimers
32.6 wt % phenylindanes
3.1 wt % linear α-methylstyrene dimers
7.7 wt % mixed alkyl-arene trimers

### Example 6

The procedure of Example 3 is followed, using 75 grams of diisobutylene and 225 grams of α-methyl-styrene, 300 grams of cumene, and 6 grams of Amberlyst™ 35 catalyst. The mixture is held for 3 hours at 55° C. after monomer addition is complete. GC/MS analysis shows a composition comprising:
2.6 wt % unstripped solvent/monomer residues.
0.6 wt % unreacted α-methyl-styrene.
3.0 wt % diisobutylene and mixed aliphatic dimers
43 wt % diisobutylene/α-methylstyrene cross dimers
31 wt % phenylindanes
12 wt % linear α-methylstyrene dimers
8 wt % mixed alkyl-arene trimers

### Example 7

The procedure of Example 1 is followed, using 75 grams of diisobutylene and 225 grams of α-methyl-styrene, 162 grams of cumene, and 6 grams of Amberlyst™ 35 catalyst. The reaction mixture is held for 2 hours at 95° C. after monomer addition is complete. GC/MS analysis shows a composition comprising:
1.3 wt % unstripped solvent/monomer residues.
1.5 wt % unreacted α-methylstyrene.
7.5 wt % diisobutylene and mixed aliphatic dimers
33 wt % diisobutylene/α-methylstyrene cross dimers
46 wt % phenylindanes
6.6 wt % linear α-methylstyrene dimers
4.2 wt % mixed alkyl-arene trimers

### Example 8

Condensation of α-methylstyrene and diisobutylene is carried out in bulk, by charging 75 grams of diisobutylene, 225 grams of α methyl-styrene, 162 grams of cumene to a reaction flask, heating the mixture to 55° C. with good stirring, and adding 6 grams of Amberlyst™ 35 catalyst at that temperature. The mixture is held for 2 hours at 55° C. after catalyst addition, then analyzed by GC/MS, which shows a composition comprising:
2.5 wt % monomer residues.
0.5 wt % unreacted α-methylstyrene.
1.5 wt % diisobutylene and mixed aliphatic dimers
41.7 wt % diisobutylene/α-methylstyrene cross dimers
31.5 wt % phenylindanes
11.8 wt % linear α-methylstyrene dimers
10.5 wt % mixed alkyl-arene trimers

### Example 9

The procedure of Example 1 is followed, using 38 grams of diisobutylene and 113 grams of α-methylstyrene, 600 grams of cumene, and 3.0 grams of an aluminum-molybdenum-phosphorous heteropolyacid catalyst. The mixture is held for 1 hour at 60° C. after monomer addition is complete, then analyzed by GC/MS, which shows a composition comprising (exclusive of about 26% unreacted monomers, not reported):
9.5 wt % diisobutylene/α-methylstyrene cross dimers
11.2 wt % phenylindanes
70.9 wt % linear α-methylstyrene dimers
6.0 wt % mixed alkyl-arene trimers

### Example 10

The procedure of Example 1 is followed, using 38 grams of diisobutylene and 113 grams of α-methylstyrene, 600 grams of cumene, and 3.0 grams of an aluminum-molybdenum-phosporous heteropolyacid catalyst. The mixture is held for 1.5 hour at 55° C. after monomer addition was complete, then analysed by GC/MS, which shows a composition comprising:
15.0 wt % unreacted diisobutylene
3.3 wt % unreacted α-methylstyrene
8.8 wt % diisobutylene/α-methylstyrene cross dimers
9.8 wt % phenylindanes
58.5 wt % linear α-methylstyrene dimers
4.6 wt % mixed alkyl-arene trimers

### Example 11

The same equipment setup as in Example 1 is used. The flask is charged with 738 grams of cumene and 15 grams of dry Amberlyst™ 35 catalyst, and stirred while heating to 55° C. under a slow nitrogen purge. A monomer mixture comprising 224 grams of diisbutylene, 354 grams of (α-methylstyrene, and 160 grams of 4-tert-butylstyrene is added rapidly drop-wise over 2 hours with good stirring, while maintaining the temperature at 55-60° C. The mixture is stirred at 55-60° C. for 4 hours after the monomer addition is complete, the catalyst is removed by filtration, and the filtrate is analyzed by GC/MS, which shows it to comprise:
2.0 wt % residual monomers.
3.6 wt % diisobutylene dimeric components.
46 wt % mixed olefin/arene cross-dimers.
30 wt % mixed cross-trimeric components.
18.5 wt % higher mixed oligomers.

### Example 12

The same equipment setup as in Example 1 is used, but the procedure is modified as follows: A monomer/solvent mixture is prepared, comprising 369 grams of cumene, 112 grams of diisobutylene, 177 grams of α-methylstyrene, and 80 grams of 4-tert-butylstyrene. The reaction flask is charged with ⅓ of this monomer/solvent mixture, and the temperature is adjusted to 55° C. with good stirring under a slow nitrogen purge. Dry Amberlyst™ 35 catalyst (7.5 grams) is added with stirring, whereupon the temperature rises to 85° C. The mixture is stirred and allowed to cool to 55° C., and the remaining monomer/solvent mixture is added dropwise in a steady stream over 2 hours at 55-60° C. The reaction mixture is held at 55° C. for 1 hour after monomer addition is complete, then filtered to remove catalyst, and analyzed by GC/MS, which shows a composition comprising:
4.0 wt % diisobutylene dimeric compoments.
50.2 wt % mixed olefin/arene cross-dimers.
28.6 wt % mixed cross-trimeric components.
17.2 wt % higher mixed oligomers.

### Example 13

The same equipment setup as in Example 1 is used. The 2-liter 4-neck flask is charged with 620 grams of cumene and 14.8 grams of dry Amberlyst™ 35 catalyst, and stirred while heating to 55° C. under a slow nitrogen purge. Then, 236 grams (2 moles) of α-methylstyrene is added rapidly dropwise over 1 hour with good stirring, while maintaining the temperature at 55-60° C. A monomer mixture comprising 224 grams (2 moles) of diisobutylene and 118 grams (1 mole) of α-methylstyrene in 115 grams of cumene is then added without pause in a rapid dropwise manner, at a steady rate over 30 minutes at 55-60° C., followed immediately by a third monomer addition of 160 grams (1 mole) of 4-tert-butylstyrene over 30 minutes. The mixture is stirred at 55-60° C. for 2 hours, then vacuum-stripped at 135° C./1.3 kPa (10 mm Hg), to yield 561 grams of straw-colored residue, having a kinematic viscosity of 2.9 mm²/s (cSt) at 100° C., and a dynamic viscosity of 60,000 cP at −30° C. Analysis of the product indicates that it comprises
8.2 wt % residual monomers.
11.6 wt % diisobutylene dimeric components.
57.6 wt % mixed olefin/arene cross-dimers.
21.6 wt % mixed cross-trimeric components.
<1 wt % higher oligomers.

### Example 14

The procedure of Example 1 is followed, using 168 grams of diisobutylene, 240 grams of 4-tert-butylstyrene, and 408 grams of cumene, with monomer addition over 2 hours and a hold time of 4 hours at 105° C. after monomer addition is complete. Vacuum-stripping yields 400 grams of straw-colored residue having a kinematic viscosity of 5.7 mm²/s (cSt) at 100° C., which, by GC/MS analysis, shows a composition comprising:
2.0 wt % residual monomer.
20 wt % diisobutylene dimeric components.
55.2 wt % mixed olefin/arene cross dimers.
22.3 wt % mixed cross-trimeric components.

### Example 15

A mixture of 84 grams of diisobutylene, 240 grams of 4-tert-butylstyrene, and 324 grams of cumene is charged to a 1-liter flask, and heated to 75° C. with stirring. Amberlyst™ 35 (6.5 grams) is added, whereupon the temperature rise to 100° C. The mixture is held with stirring at 100° C. for 4 hours, then at 105° C. for 6 hours. The catalyst is removed by filtration, and the filtrate is vacuum-stripped at 120° C./2.7 kPa (20 mm Hg) pressure, to yield 289 grams of straw-colored residue.

### Example 16

The procedure of Example 1 is followed, using 140 grams of diisobutylene, 260 grams of styrene, 400 grams of cumene, and 8 grams of Amberlyst™ 35. The mixture of monomers is added over 2 hours at 105° C., and the mixture held for 4 hours at 105° C. after monomer addition is complete. An exotherm of 7° C. is observed during the initial stages of monomer addition. Catalyst and cumene solvent are removed, and GC/MS analysis of the reaction mixture residue shows it to comprise:
8% styrene.
16% residual diisobutylene and lower alkyl benzenes.
4% diisobutylene dimers.
52% styrene-olefin cross-dimeric components.
20% styrene-olefin cross-trimeric components.

### Example 17

The procedure of Example 1 is followed, using 151 grams of propylene tetramer, 212 grams of α-methylstyrene, 363 grams of cumene, and 7.3 grams of Amberlyst™ 35 catalyst, with a monomer addition time of 1.5 hours, and a hold time of 4.5 hours at 105° C. after monomer addition is complete.

### EXAMPLES 18-19

### Hydrogenation

### Example 18

The reaction mixture from Example 5, after vacuum-stripping, is hydrogenated in an autoclave using a supported rhodium catalyst at 3.4 MPa (500 psig) hydrogen and approximately 150° C. for a time of approximately 12 hours, resulting in approximately 95% removal of aromatic and olefinic unsaturation. The hydrogenated product has a kinematic viscosity of 2.53 mm²/s (cSt) at 100° C., with a dynamic viscosity at −30° C. of 3.65 Pa-s (3,650 cP) and a traction coefficient of 0.092.

### Example 19

The reaction mixture from Example 13, after vacuum-stripping and hydrogenation as in Example 18 to substantially remove olefinic and aromatic components, has a kinematic viscosity 4.0 mm 2/s (cSt) at 100° C., and 24.5 mm²/s (cSt) at 40° C., a dynamic viscosity at −30° C. of 67.0 Pa-s (67,000 cP), and a traction coefficient of 0.095.

### EXAMPLES 20-21

### Example 20

Example 5 is substantially repeated on approximately an 8-fold larger scale, providing a fluid which is subsequently hydrogenated as in Example 18, except for a period of about 20 hours. The kinematic viscosity of the product is 2.79 mm²/s (cSt) at 100° C. and 11.84 mm²/s (cSt) at 40° C. Its dynamic viscosity at −30° C. is 2.33 Pa-s (2,330 cP).

### Example 21

Example 13 is substantially repeated on an approximately 8-fold larger scale, providing a fluid which is subsequently hydrogenated as in Example 20. The kinematic viscosity of the product is 4.43 mm²/s (cSt) at 100° C. and 28.9 mm²/s (cSt) at 40° C. Its dynamic viscosity at −30° C. is 42.0 Pa-s (42,000 cP).

The traction coefficients of the products of Examples 20 and 21, as well as that of a commercial traction fluid base fluid, are measured at 1.25 GPa pressure, 4 m/s rolling velocity, at slide/roll ratios up to 10%, at various temperatures. In all cases the plot of traction coefficient versus slide/roll ratio % is approximately flat between 2% and 10% slide/roll ratio. Values at 6% ratio at various temperatures are presented in the following Table 1:

TABLE 1

| Temperature | Example 20 | Example 21 | Commercial (ref) |
|---|---|---|---|
| 50° C. | 0.104 | 0.106 | 0.116 |
| 75° C. | 0.095 | 0.100 | 0.108 |
| 100° C. | 0.084 | 0.092 | 0.097 |
| 120° C. | 0.075 | 0.083 | 0.086 |

### EXAMPLES 22-29

### Condensation of Terpenes and Olefins

### Example 22

To a 4-neck, 500 mL flask fitted with a nitrogen inlet, thermowell, stirrer, and cold water condenser vented to a dry ice trap, the following ingredients are charged: 170 g limonene, 70 g diisobutylene, 14 g toluene, and 4.8 g Amberlyst™ 15 catalyst. The mixture is heated with medium stirring to 100° C. and held at temperature for six hours, under a nitrogen sweep of 11 L/hr (0.4 std. ft³/hr). The product is vacuum stripped at 100° C., 1.7 kPa (30 mm Hg) to remove toluene and unreacted starting materials and is filtered to remove residual catalyst. The product is a clear yellow liquid.

### Example 23

Example 22 is substantially repeated except that the amounts are: limonene, 204 g; diisobutylene, 56 g; toluene, 52 g; Amberlyst™ 15, 7.8 g. The mixture is heated to 120° C. rather than 100° C. The product is a clear yellow liquid.

### Example 24

Example 23 is substantially repeated except that the mixture is heated to 80° C. The product is a clear yellow liquid.

### Example 25

Example 23 is substantially repeated except that a 5 L flask is used and the amounts of materials are: limonene, 1224 g; diisobutylene, 336 g; toluene, 312 g; Amberlyst™ 15, 31.2 g. The product is a clear orange liquid.

### Example 26

To a 2 L flask, equipped as in Example 22, the following materials are added: limonene, 272 g; diisobutylene, 224 g; cyclohexane, 496 g; a heteropolyacid catalyst, 10 g The mixture is heated at 60° C. for 2 hours with no appreciable reaction; thereafter it is heated to 990° C. for 6 hours. The product, after workup as in Example 20, is a clear yellow liquid.

### Example 27

Example 1 is substantially repeated, using 136 g limonene, 112 g diisobutylene, 50 g cyclohexane, and 5.0 g heteropolyacid catalyst (60% active catalyst) on silica support. In this example the diisobutylene is fed dropwise over 1/2 hour after the mixture is heated to 100° C.

### Example 28

Example 1 is substantially repeated in a 1 L flask, using 272 g limonene, 112 g diisobutylene 38 g cumene, and 7.7 g Amberlyst™ 35 catalyst. The limonene and diisobutylene are added dropwise to the mixture over 2 hours, at 120° C., and the mixture is held at temperature for 6 hours. The product is a yellow liquid which is analyzed and found to contain:
5.93% diisobutylene dimer
22.64% cross dimer
50.35% terpene dimers
5.35% mixed cross trimers
15.73% terpene trimers

### Example 29

Example 1 is substantially repeated in a 2 L flask, using 544 g limonene, 224 g diisobutylene, 329 g cumene, and 15.36 g Amberlyst™ 35 catalyst. The mixture is heated to 120° C. until reflux ceases, then heated to 135° C. and held for 4 hours. Analysis of the unstripped product indicates 92% conversion, providing:
12% diisobutylene dimers,
22% terpene/diisobutylene dimer,
33% terpene dimers,
26% trimer of terpene+2 diisobutylene, and
7% mixture of terpene trimer and trimer of 2 terpenes/diisobutylene.

The products of Examples 21-29 are hydrogenated using a process similar to that of Example 18, except that a nickel-on alumina catalyst is used and the time of reaction is 6-8 hours, at 400 psig hydrogen pressure.

### Example 30

A fully formulated traction fluid composition is prepared by blending 30 parts by weight of the product of Example 20 with 70 parts of the commercially available traction fluid base fluid used for comparison in Table 1. Added to the blend is 3.85 percent by weight of a conventional automatic transmission fluid additive package containing one or more detergents, dispersants, antiwear agents, and antioxidants, along with diluent oil and other minor components. Also added is 3% of a commercial polymethacrylate viscosity modifier composition. The viscosity and traction coefficient performance characteristics are reported in Table 2, below.

TABLE 2

| | | | Dynam- | | | | | |
|---|---|---|---|---|---|---|---|---|
| | Kinematic | | ic Vis- | | | | | |
| | Viscosity, | | cosity, | Traction Coefficient at 100° C., | | | | |
| | mm²/s | | Pa-s | 4.0 m/s Slide/Roll Ratio, % | | | | |
| | 100° C. | 40° C. | −30° C. | 1% | 2.5% | 5% | 7.5% | 10.0% |
| Ex 30 | 4.21 | 21.76 | 17.4 | 0.079 | 0.086 | 0.089 | 0.089 | 0.089 |

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent oil, which may be customarily present in the commercial material, unless otherwise indicated. It is to be understood that the upper and lower amount, range, and ratio limits set forth herein may be independently combined. Similarly, the ranges and amounts for each element of the invention can be used together with ranges or amounts for any of the other elements. As used herein, the expression “consisting essentially of” permits the inclusion of substances that do not materially affect the basic and novel characteristics of the composition under consideration.

1. A composition suitable for use as a traction fluid, comprising a substantially completely hydrogenated addition product of
(a) at least one olefin monomer containing a cyclic hydrocarbon structure, with
(b) at least one non-cyclic olefin monomer of at least 4 carbon atoms, provided that if the non-cyclic olefin monomer is monounsaturated, then it contains at least 5 carbon atoms.

2. The composition of claim 1 wherein said hydrogenated addition product is a dimer or oligomer comprising up to about 10 total units of monomers (a) and (b).

3. The composition of claim 1 wherein said hydrogenated condensation product has a Brookfield viscosity of less than about 70 Pa-s (70,000 cP) at −30° C.

4. The composition of claim 1 wherein the olefin monomer of (a) contains a vinyl group which (i) is attached to an aromatic ring or which (ii) bears an α-substituent.

5. The composition of claim 1 wherein component (a) is a vinyl arene.

6. The composition of claim 5 wherein the vinyl arene is styrene, α-methylstyrene, or a ring-alkylated styrene.

7. The composition of claim 1 wherein component (a) is a cyclic terpene.

8. The composition of claim 7 wherein the cyclic terpene is α-pinene, β-pinene, limonene, α-terpinene, β-terpinene, or β-phellandrene.

9. The composition of claim 1 wherein the non-cyclic olefin monomer (b) contains 1, 2, or 3 ethylenic double bonds.

10. The composition of claim 1 wherein the non-cyclic olefin monomer (b) is a non-cyclic monomer selected from the group consisting of linear and branched hexenes, linear and branched octenes, linear and branched decenes, propylene trimers, propylene tetramers, and isobutylene dimers, trimers, and tetramers.

11. The composition of claim 1 wherein the non-cyclic olefin monomer (b) is isoprene or 1,3-hexadiene.

12. The composition of claim 1 wherein the non-cyclic olefin (b) is a non-cyclic terpene.

13. The composition of claim 1 wherein components (a) and (b) each comprise about 10 percent to about 90 percent by weight of the total of all monomers present in the addition product.

14. The composition of claim 1 wherein (a) is at least one vinyl aromatic monomer and (a) comprises 40 to 80 weight percent of all monomers present in the addition product, and (b) comprises 60 to 20 weight percent of all such monomers.

15. The composition of claim 1 wherein said fluid is prepared by the acid-catalyzed addition reaction of the monomers of (a) and (b).

16. The composition of claim 15 wherein said addition reaction is conducted in the presence of a solvent.

17. The composition of claim 1 further comprising at least one additive selected from the group consisting of dispersants, detergents, friction modifiers, antioxidants, metal passivators, viscosity modifiers and antiwear agents in an amount sufficient to improve the performance of said composition in a power transmission device.

18. The composition of claim 1 further comprising an oil of lubricating viscosity other than said hydrogenated addition product.

19. The composition of claim 1 further comprising at least one additional traction fluid.

20. The composition of claim 1 comprising a plurality of said hydrogenated addition products having differing viscosities.

21. A method for lubricating a power transmission apparatus, comprising employing therein the composition of claim 1.

22. A method for preparing a composition suitable for use as a traction fluid, comprising:
(a) combining
(i) at least one olefin monomer containing a cyclic hydrocarbon structure, with
(ii) at least one non-cyclic olefin monomer of at least 4 carbon atoms, provided that if the olefin monomer is monounsaturated, then it contains at least 5 carbon atoms; and
(iii) an acid catalyst;
(b) maintaining the resulting mixture at about 25° C. to about 150° C. for a time sufficient to permit reaction of components (a)(i) and (a)(ii);
(c) optionally removing the volatile components from the product of (b); and
(d) substantially completely hydrogenating the resulting reaction product.

23. The method of claim 22 wherein the acid catalyst is a sulfur acid, a phosphorus acid, or a halogen acid.

24. The method of claim 22 wherein the acid catalyst of (iii) is a heteropolyacid in its acid, salt, or partially salted form.
